(19) United States
(12) Patent Application Publication
(10) Pub. No.: US 2009/0247955 A1
(43) Pub. Date: Oct. 1, 2009

(54) MICROLITER INJECTOR

(75) Inventors: Ronald K. Yamamoto, San Francisco, CA (US); Stanley R. Conston, San Carlos, CA (US)

Correspondence Address: Weaver Austin Villeneuve & Sampson LLP, P.O. BOX 70250, OAKLAND, CA, 94612-0250, US

(73) Assignee: iScience Interventional Corporation, Menlo Park, CA (US)

(21) Appl. No.: 12/412,586

(22) Filed: Mar. 27, 2009

Related U.S. Application Data
(60) Provisional application No. 61/040,009, filed on Mar. 27, 2008.

(51) Int. Cl. A61M 5/31

(52) U.S. Cl. 604/201

(57) ABSTRACT

Injectors are provided that may be pre-filled aseptically with an active biological agent into a sterile cavity and sealed in such a manner that the injectate may be readily expelled into the body. The injectors are particularly useful for injecting precisely measured small volumes of sterile agents.

### CROSS-REFERENCE TO RELATED APPLICATION

The priority of provisional U.S. application Ser. No. 61/040,009, filed Mar. 27, 2008 is claimed pursuant to 35 USC 119(e). The provisional application is incorporated herein by reference in its entirety.

### BACKGROUND OF INVENTION

Many therapeutic agents in medicine are effective in extremely small doses. Current pharmaceutical practice is to dilute the agents with excipients in order to have a volume sufficient to deliver into a body through standard means such as a hypodermic needle and syringe or percutaneous catheter. The smallest commercially available syringes are on the order of 250 microliter volume and have insufficient accuracy to provide for precise volumetric delivery of injectates below 100 microliters. Microliter syringes are available for use in analytical chemistry applications (e.g. Hamilton syringes), but are not designed or indicated for use in clinical applications. With the advent of biopharmaceutical, gene therapy and other highly active therapeutic agents, it is desirable to be able to precisely inject small, e.g. nanoliter or microliter, amounts of agents into the body. One such application is the injection of biopharmaceutical agents into the vitreous cavity of the eye to treat diseases such as diabetic retinopathy or macular degeneration.

It is further desirable to provide means to have a pre-filled injection device to provide a precise pre-measured amount of a flowable injectate for administration. Such means require that the injectate in the device be filled in an aseptic manner or be able to be terminally sterilized. The injectate may be sealed within the device until ready for use. It is also an objective to aid the delivery precision by minimizing the potential dead space in the flow path of the injectate.

The present invention describes a device which would allow for aseptically filling of an injectate into a sterile cavity and the sealing of said cavity in such a manner that the injectate can be easily expelled for delivery into a body. The cavity is preferably incorporated into a manual plunger syringe with an attached or incorporated hypodermic needle for injection into the body. The filled device may be terminally sterilized to provide a pre-filled, ready to use injector for the user. The design of the device is such that it may be sized to provide injection volumes from hundreds of nanoliters to hundreds of microliters.

### SUMMARY

In one embodiment, an injector device is provided comprising: a container having a proximal end and a distal end and being made of a substantially rigid material. The container has an elongated cavity extending therethrough from the proximal end to the distal end, and an opening at the distal end for discharging injectate from the cavity. A rupturable seal is located at the distal end to prevent injectate contents of the cavity from exiting through the opening. The injector has a displacement body slideably located to traverse the cavity from the proximal end to the distal end and is thereby capable of applying compressive pressure on the contents of the cavity sufficient to rupture the seal to eject at least a portion of the injectate from the cavity. In another embodiment, a sliding element within the cavity actuated by the displacement body may rupture the seal to initiate delivery of the injectate. The displacement body may preferably be a piston or rod.

The displacement body generates pressure in the container sufficient to rupture the rupturable seal without piercing the seal by contact.

The injector may further comprise a pointed needle at the distal end having a passage through which fluid contents (injectate) in the cavity exit the injector upon rupture of the seal. As an alternative to the needle the distal end of the container may be pointed and have a passage through which fluid contents in the cavity are ejected upon rupture of the seal.

In another embodiment, an injector is provided comprising:

a container having a proximal end and a distal end, the container having an elongated cavity extending therethrough, a first opening to the cavity at the distal end for discharging injectate from the container; a second opening to the cavity at the proximal end for slidably accommodating a hollow rod; a first rupturable seal and a second seal, respectively covering the first and second openings to prevent the injectate from exiting the cavity;

a hollow rod having a pointed distal end and a proximal end, a longitudinal passage with a distal opening at the pointed end, a proximal opening located on the side of the rod, and a marker at the proximal end of the rod, such as a handle, flange, notch, or other marker to stop the insertion, whereby the maximum length of insertion of the rod into the container through the second opening of the cavity and second seal into the cavity and through the first opening of the cavity and first seal is provided by the marker. At the maximum length of insertion of the rod through the container from the second end to the first end, the pointed distal end of the rod protrudes from the container at the distal end of the container and the proximal opening of the rod on the side of the rod is located within the cavity proximal to the first opening.

Before use of the injector, the rod may be affixed to the container by partial insertion into the second opening without rupturing the second seal. The injector may be provided as a kit in which the container and rod are separately provided and assembled when ready for use. The container may be at least partially made of a flexible material that may be squeezed to provide pressure on injectate contents of the cavity to thereby eject the injectate contents via the proximal opening on the side of the rod. Alternatively, the injectate may be sealed within the container under pressure, whereby the injectate is ejected when the proximal opening in the rod comes into communication with the injectate under pressure in the cavity.

An injector according to the invention is advantageously adapted for incorporation into aseptic packaging in a kit wherein the injector contains an injectate sealed within the cavity for use.

### BRIEF DESCRIPTION OF THE DRAWING

FIG. 1: Schematic of a device incorporating a cavity within a syringe body.

FIG. 2: Schematic of a device incorporating a cavity within an injection needle.

FIG. 3: Schematic of a device incorporating a cavity with proximal and distal seals, and a fenestrated piercing needle.

### DESCRIPTION OF THE PREFERRED EMBODIMENTS

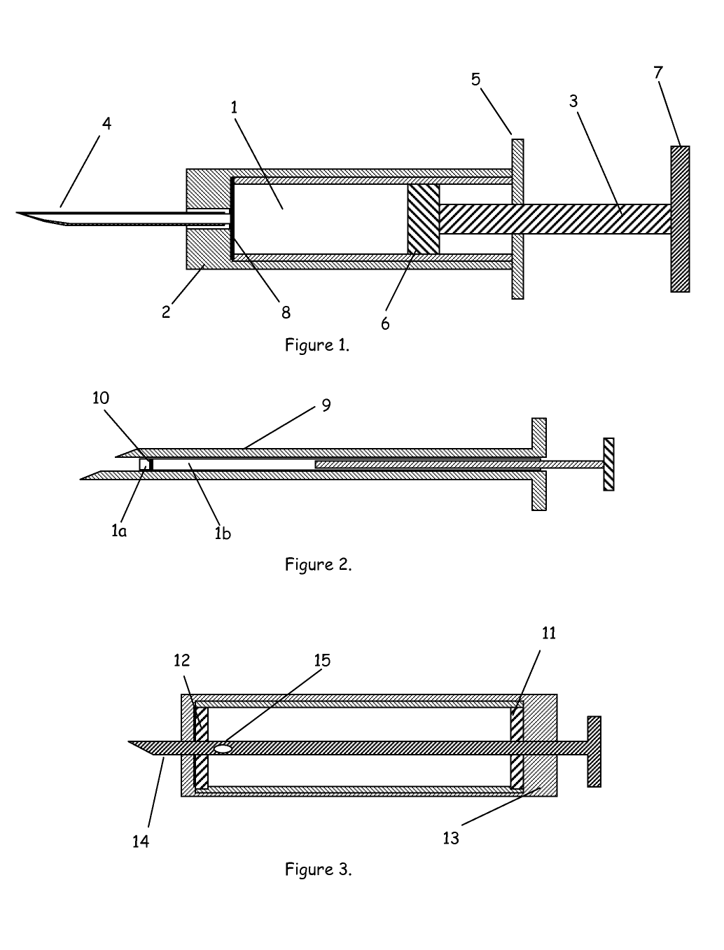

The present invention provides devices for delivering nano- or micro-liter quantities of a therapeutic agent formulated as a fluid or a flowable material in a pre-filled, ready to use, sterile device for injection into a subject's body or body cavity. Referring to FIG. 1, an embodiment of device according to the invention comprises a cavity 1, body 2, plunger 3 and may optionally include a hypodermic needle 4.

The cavity 1 resides within the body of the device. The body may be fabricated such that the body comprises the cavity as a feature or the cavity may be fabricated as a separate component which is assembled into the body. The proximal end of the body may incorporate a flange or flanges 5 for placement of the user's fingers to support the body during injection. The flanges may be in the form of a simple cross-bar or may incorporate finger holes for additional manual control.

Preferably, a hypodermic needle 4 is incorporated into the distal end of the body for piercing and injection into the subject's body. Alternately, a male Luer or Luer-lock fitting may be incorporated onto the distal end of the body for placement of a conventional, commercially available hypodermic needle. For example, to inject agents into the vitreous cavity of the eye, a needle size of 27 gauge or smaller is preferred, such that the injection site is self-sealing, i.e. it does not require suturing to close.

The plunger 3 is axially disposed within the lumen of the cavity 1 and is used to express the injectate out of the device. The plunger may comprise a distal piston 6 to provide for direct sealing of the cavity lumen to prevent injectate from leaking around the plunger. Alternatively, the plunger itself may provide the pressure to rupture a seal as shown in FIG. 2.

In another embodiment, the plunger acts upon a moveable seal in addition to or in place of piston 6 disposed in the proximal end of the cavity lumen. The moveable seal is slideable within the cavity, but is not intended to be rupturable under pressure of the plunger. The proximal end of the plunger preferably incorporates a flange or button 7 to facilitate depression of the plunger by the user. In a preferred embodiment, the plunger incorporates a stop mechanism (such as piston 6 contacting the wall accommodating flanges 5) to prevent the plunger from being removed from the body 2. Furthermore, the plunger may incorporate a locking mechanism (not shown) to prevent the plunger from being prematurely activated.

The cavity 1 comprises a cylindrical member with proximal and distal ends composed of an inert material such as glass, plastic, fluoropolymer, or passivated metal. The cavity may be designed as a separate component which is assembled into the body 2, or alternatively, the body 2 may be designed such that the cavity is an integral part thereof. An example of a separate cavity is a tube to act as the reservoir for the injectate, which is placed inside the body 2 of the device. An example of an integral cavity is a fluoropolymer coating forming a chemically inert reservoir area within the body lumen. The material of the cavity 1 is chosen to provide a compatible and inert surface, such as glass or passivated glass, for contacting the injectate contained therein. The distal end of the cylindrical cavity 1 is sealed across the lumen with a thin seal or septum 8, which may comprise materials such as low density polyethylene, metal foil or similar thin film materials. The septum 8 will have a thickness sufficient to rupture from the compressive pressure applied when the plunger 3 is depressed onto the contents of the cavity. The material of the septum will be of a material, such as a metal foil, that tears rather than shatters into fragments under pressure. It is undesirable to produce fragments of the septum that may be injected into the body or cause blockage in the needle 4. This seal 8 is fabricated or assembled onto the distal end of the cavity. The proximal end of the cavity may incorporate a cylindrical disc (not shown) of compatible polymer or elastomer, which acts to seal the cavity and which is acted upon by the plunger 3. When the plunger is depressed, the pressure generated causes the rupture of the distal seal 8, allowing delivery of the injectate. The distal seal may have partial thickness perforations or similar features to insure a clean rupture of the seal to control the configuration of the seal after rupture. Once filled, sealed and packaged, the device may be subjected to common sterilization methods to provide a single use system for the user.

The device may be sized according to the quantity of injectate desired. In the case of nanoliter volumes 1a, a device such as that shown in FIG. 2 may be fabricated into the bore of an injection needle 9, with a slideable polymer seal 10 fabricated inside the bore near the distal tip. The slideable seal 10 defines the volume 1a between it and the sealed distal end of the cavity that is to be injected into the subject. For example, a 30 gauge by one-half inch long hypodermic needle has the bore of 0.006 inches and a volume of 200 nanoliters. Diameters and lengths of the needle may be sized for the appropriate volume. In the case of larger volumes, the cavity is positioned within the body and is sized accordingly. The dead volume 1b of the flow path may be compensated for by filling the cavity with additional injectate. In the example cited above, a 30 gauge needle volume of 200 nanoliters would be added to the volume of the fill 1b to allow for delivery of the precise intended amount 1a. The solid plunger provides the pressure to move the slideable seal 10.

In another embodiment shown in FIG. 3, a cavity is fabricated such that a septum (11, 12) is used to seal the proximal and distal ends of the cavity. One septum is placed prior to filling and the other is placed after filling the cavity. The cavity is then placed into a body 13 which incorporates a slidably disposed hollow needle 14. The needle incorporates a fenestrated side hole 15 to allow injectate ingress into the bore of the needle. The pointed needle distal tip resides outside the cavity in a sterile condition in a position for shipping and storage. During use, the needle is advanced axially, piercing both the proximal and distal septa 11 and 12 of the cavity. The side hole 15 in the needle 14 is positioned such that it resides close to the distal septum 12 when fully advanced while remaining in communication with the contents of the cavity. The device furthermore is configured to incorporate a way to pressurize the cavity so as to force the injectate through the needle side hole 15, to be delivered into the subject's body through needle 14. Pressure on the contents of the cavity may be provided, for example, by making the body walls of body 13 from a flexible material so the user can squeeze it to eject injectate through side hole 15. Pressure may also be applied by loading the contents of the cavity initially under pressure. Other methods of applying pressure to a volume of fluid or flowable material may be used.

One example of the fabrication of a device according to the invention is as follows. The cavity and plunger components are fabricated and sterilized by common means. The cavity is then filled aseptically using a small diameter fill tube to fill from the bottom (proximal end) of the cavity upward, so as to prevent air bubble entrapment. A film seal is aseptically placed onto the distal end of the cavity. If fabricated as a separate component, the cavity is then placed into the body and the device assembled. The finished device is then aseptically packaged for use. Alternatively, the device may be terminally sterilized by means that will not affect the active agent which may be aided by shielding the cavity from the sterilization apparatus, gas, radiation or heat. Common sterilization includes ethylene oxide gas, electron beam or gamma irradiation methods.

### EXAMPLES

### Example 1

A 10 microliter glass capillary tube (Drummond Scientific) was used as a cavity for the injectate. The 10 microliter tube had a lumen diameter of 0.021 inches and a length of 1.62 inches. A small piece of 0.001″ thick linear low density polyethylene film (Winzen Films) was stretched across one end of the cavity and secured in place with thin walled PET heat shrink tubing (Advanced Polymers). A plunger seal was created by filling a short segment of a second capillary tube with UV cure epoxy with a durometer of 27 Shore D (Loctite). The epoxy plug was cured, removed from the tube and then trimmed into a thin cylindrical plug.

The glass cavity was filled with water using a long 34 gauge fill needle, and the epoxy plug was placed into the proximal end. A small diameter wire was inserted between the outer edge of the plug and the inner wall of the cavity to bleed the air out in the cavity while the plug was inserted fully using a 0.021″ steel pin as a plunger. Once filled and sealed, the plunger was depressed causing the polyethylene seal to burst, delivering the fluid.

### Example 2

A prototype injector was fabricated using a glass cavity as described in Example 1 above. The body and plunger were fabricated using type 304 stainless steel hypodermic tubing. The body of the device was split into two pieces to allow for the replacement of the glass cavity. The distal body incorporated a seat for the glass cavity and a 30 gauge by ½ inch hypodermic needle at the tip. The proximal body incorporated circular finger grips and a plunger assembly. The plunger assembly incorporated a stop mechanism to prevent the plunger from being removed from the proximal body. The two body sections were attached via a machined bayonet type mount.

The epoxy plug was placed into the proximal end of the glass cavity and the cavity was filled completely with water using a long fill needle. The film seal was stretched over the distal end of the cavity, sealing the fluid inside. Excess film was trimmed away and the glass cavity inserted into the distal body section. The proximal body section was attached and then the plunger was depressed, forcing the fluid out of the distal needle tip.

What is claimed is:

1. An injector comprising:
a container having a proximal end and a distal end, comprising a substantially elongated cavity extending therethrough from said proximal end to said distal end, and an opening at said distal end for discharging injectate from said cavity;
a rupturable seal located at said distal end to prevent injectate contents of said cavity from exiting through said opening; and
a displacement body slideably located to traverse said cavity from said proximal end to said distal end, to provide compressive pressure on injectate contents of said cavity sufficient to rupture said seal thereby ejecting at least a portion of said injectate contents from said cavity through said opening.

2. The injector according to claim 1 further comprising a pointed needle at said distal end communicating with said opening, said needle having a passage through which injectate contents in said cavity exit said injector upon rupture of said seal.

3. The injector according to claim 1 wherein said distal end of said container is pointed and has a passage through which fluid contents in said cavity exit said injector upon rupture of said seal.

4. The injector according to claim 1 wherein said displacement body comprises a piston.

5. The injector according to claim 1 wherein said displacement body comprises a rod.

6. The injector according to claim 1 further comprising a moveable non-rupturable seal proximal to said opening.

7. The injector according to claim 1 wherein said container comprises a rigid material.

8. An injector comprising:
a container having a proximal end and a distal end, said container having a substantially elongated cavity extending therethrough from said proximal end to said distal end, a first opening to said cavity at said distal end for discharging injectate from said container, a second opening to said cavity at said proximal end for slidably accommodating a hollow rod,
a first rupturable seal and a second rupturable seal, respectively, covering said first and second openings to prevent injectate contents from exiting said cavity;
said hollow rod having a pointed distal end, a proximal end, a passage with a distal opening at said pointed end, a proximal opening located on the side of said rod, and a marker at said proximal end of said rod whereby the maximum length of insertion of said rod into said container through said second opening of said cavity and second seal into said cavity and through said first opening of said cavity and first seal is provided by said marker;
whereby at the maximum length of insertion of said rod through said container from said second end to said first end of said container said pointed distal end of said rod protrudes from said container at said first end and said proximal opening of said rod on the side of said rod is located within said cavity proximal to said first opening.

9. The injector according to claim 8 wherein said rod is affixed to said container by partial insertion into said second opening without rupturing said second seal.

10. The injector according to claim 8 wherein said container is at least partially made of a flexible material that may be squeezed to provide pressure on fluid contents of said cavity to thereby eject said injectate contents via said proximal opening on the side of said rod.

11. A kit comprising an aseptically packaged injector according to any one of claims 1 through 7 containing an injectate sealed within said cavity.

12. A kit comprising an aseptically packaged injector according to any one of claims 8 through 10 containing an injectate sealed within said cavity.
